(19) United States
(12) Patent Application Publication
(10) Pub. No.: US 2025/0213336 A1
(43) Pub. Date: Jul. 3, 2025

(54) METHODS FOR CONDUCTING GUIDED ORAL AND MAXILLOFACIAL PROCEDURES, AND ASSOCIATED SYSTEM

(71) Applicant: NEOCIS INC., Miami, FL (US)

(72) Inventor: Alon Mozes, Miami Beach, FL (US)

(21) Appl. No.: 19/087,212

(22) Filed: Mar. 21, 2025

Related U.S. Application Data
(63) Continuation of application No. 16/665,980, filed on Oct. 28, 2019.
Application No. 16/665,980 is a continuation of application No. PCT/IB2018/052955, filed on Apr. 27, 2018.
(60) Provisional application No. 62/491,410, filed on Apr. 28, 2017.

(51) Int. Cl. A61C 13/00; A61B 5/00; A61B 34/00; A61B 34/10; A61B 34/30; A61B 90/00; A61C 1/00; A61C 1/08; A61C 3/02; A61C 5/00

(52) U.S. Cl. CPC A61C 13/0004; A61B 5/0088; A61B 34/10; A61B 34/30; A61B 34/76; A61B 90/39; A61C 1/0007; A61C 1/082; A61C 3/02; A61C 5/00; A61B 2090/363; A61B 2090/3937

(57) ABSTRACT

Methods of conducting guided oral or maxillofacial procedures involve forming a secure, physical interaction between a fiducial marker and a site having an associated object. A virtual plan is formed, detailing the procedure on the object or the site, in registration with the fiducial marker. Movement of a procedure-conducting device is physically regulated with a robot arm responsive to a controller device and with respect to the fiducial marker. The procedure-conducting device interchangeably receives procedural devices and a calibration element. The controller device cooperates with the robot arm to physically regulates movement of the procedure-conducting device, according to the virtual plan and corresponding with physical manipulation of the procedure-conducting device by the user, for the procedural device to conduct the procedure. Tactile feedback is provided to the user, via the procedure-conducting device, if the physical manipulation by the user causes the procedure-conducting device to deviate from the virtual plan.

### CROSS-REFERENCE TO RELATED APPLICATIONS

This application is a continuation of U.S. patent application Ser. No. 16/665,980; filed Oct. 28, 2019, which is a continuation of International Application No. PCT/IB2018/052955, filed Apr. 27, 2018, which International Application was published by the International Bureau in English on Nov. 1, 2018, and claims priority to U.S. Provisional Application No. 62/491,410, filed Apr. 28, 2017, all which are incorporated herein by reference in their entirety and for all purposes.

### BACKGROUND OF THE DISCLOSURE

### Field of the Disclosure

The present disclosure relates to dental or oral and maxillofacial procedures and, more particularly, to methods for a conducting guided oral and maxillofacial procedures, including wisdom tooth ablation, root canals, and tooth preparation for crown placement, and an associated system.

### Description of Related Art

Many oral/maxillofacial/dental procedures are manually conducted by a surgeon. In some instances, the surgeon may be assisted by a jig or other hardware element interacting with the surgical tool for the purpose of physically aligning and orienting the surgical tool to perform the procedure.

For example, many wisdom teeth (e.g., 3^{rd} molars) are generally removed in a surgical procedure. In an alternative procedure, the buds of the wisdom teeth can be ablated before they grow into full teeth, thereby negating the need for surgery. However, this is still an invasive procedure. Generally, the process requires accurate drilling of an access hole through gum tissue, etc. to access the bud in the correct location for the placement of a radioactive seed or direct a probe for ablating the bud. In one instance, the guidance of the probe requires the fabrication of a plastic surgical guide physically engaged between the probe and the site at which the procedure is to be conducted. Such a guide, however, may impede access to the site, particularly considering that the site is in the posterior of the mouth where the 3^{rd} molars are located.

In another example, a root canal procedure involves drilling a hole in a tooth to access the pulp/nerve within the roots of the tooth. Once accessed, the pulp/nerve within the root is filed or abraded out of the roots. The hollowed tooth is then filled with a filler material so that the tooth can remain intact without any nerve pain. In such a procedure, the roots of the tooth can be visible on a CT scan, but may be difficult to locate on the patient. In some instances, the dentist can fabricate a plastic surgical guide, based on the CT scan, for physically guiding the drill for drilling the initial hole in the tooth to access the interior of the roots. However, depending on the location of the tooth within the mouth of the patient, the plastic surgical guide may impede access to the site or otherwise be cumbersome to implement.

Yet another example includes preparation of a tooth for crown placement. That is, when a cavity in a tooth is sufficiently advanced, a dentist may remove a portion of the tooth and replace the removed portion with a porcelain (or zirconium, etc.) crown. This process for preparing the tooth to receive the crown requires dexterity on the part of the dentist to remove the tooth material, and high accuracy and precision for matching the crown to replace the removed tooth material.

Thus, there exists a need for a method and system for providing improvements for conducting particular oral and maxillofacial procedures that addresses the noted shortcomings of current procedures, and facilitates, for example, unimpeded access to the site of the procedure and guidance of the surgeon/appropriate instrument during the procedure without the use of a jig or other physical guide.

### BRIEF SUMMARY OF THE DISCLOSURE

The above and other needs are met by the present disclosure which, in one aspect, provides a method for conducting a guided oral or maxillofacial procedure. Such a method comprises securing a fiducial marker included with a fiducial device in a secure and physical interaction with an oral or maxillofacial site having an oral or maxillofacial object associated therewith; calibrating a calibration element in an oral or maxillofacial procedure-conducting device, the calibration element being interchangeable with a plurality of oral or maxillofacial procedural devices in the oral or maxillofacial procedure-conducting device, the plurality of oral or maxillofacial procedural devices including an ablation probe, a cutting device, a drilling device, and an abrading device, interchangeable in the oral or maxillofacial procedure-conducting device, to register any one of the plurality of interchangeable oral or maxillofacial procedural devices with the fiducial marker upon replacement thereof in the oral or maxillofacial procedure-conducting device; forming, using a procedure planning device, a virtual procedure plan detailing an oral or maxillofacial procedure at the oral or maxillofacial site, the virtual procedure plan including a procedural route of the oral or maxillofacial procedure-conducting device comprising one of the plurality of oral or maxillofacial procedural devices registered with the fiducial marker, for the one of the plurality of oral or maxillofacial procedural devices to perform the oral or maxillofacial procedure, the virtual procedure plan being in registration with the fiducial marker; upon the virtual procedure plan detailing the oral or maxillofacial procedure comprising an ablative object removal procedure at the oral or maxillofacial site, placing the ablation probe in the oral or maxillofacial procedure-conducting device; controlling, using a controller device in communication with the procedure planning device, orientation and distance of the oral or maxillofacial procedure-conducting device along a manipulation path, in correspondence with physical manipulation of the oral or maxillofacial procedure-conducting device along the procedural route by the user for the ablation probe to perform the ablative object removal procedure at the oral or maxillofacial site; and physically regulating movement of the oral or maxillofacial procedure-conducting device comprising the ablation probe, using a guidance device including a robotic arm engaged with the oral or maxillofacial procedure-conducting device, in response to the controller device in communication therewith and with respect to the fiducial marker, in accordance with the virtual procedure plan, the oral or maxillofacial procedure-conducting device providing tactile feedback to the user, if the physical manipulation of the oral or maxillofacial procedure-conducting device by the user along the manipulation path causes the oral or maxillofacial procedure-conducting device to deviate from the procedural route of the virtual procedure plan.

Various other aspects of the present disclosure may include and be directed to systems for facilitating the disclosed methods of conducting an oral and/or maxillofacial procedure. These and other features, aspects, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended to be combinable, unless the context of the disclosure clearly dictates otherwise. Aspects of the present disclosure thus provide apparent advantages as otherwise detailed herein.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 schematically illustrates a system for conducting a guided oral and maxillofacial procedure, according to one aspect of the present disclosure;

FIGS. 2 and 3 schematically illustrate a system for conducting a guided oral and maxillofacial procedure, according to an alternate aspect of the present disclosure;

FIG. 4 schematically illustrates a system for conducting a guided oral and maxillofacial procedure, according to a further aspect of the present disclosure;

FIG. 5 schematically illustrates portions of a procedure for conducting a guided oral and maxillofacial procedure, particularly for conducting a guided wisdom tooth removal procedure using an ablation probe for ablating the wisdom tooth bud, according to one aspect of the present disclosure;

FIGS. 6A-6D schematically illustrates a portion of a procedure for conducting a guided oral and maxillofacial procedure, particularly for conducting a guided root canal procedure, according to one aspect of the present disclosure; and

FIGS. 7A and 7B schematically illustrate a portion of a procedure for conducting a guided oral and maxillofacial procedure, particularly for conducting a tooth preparation procedure for the tooth to receive a crown, according to an alternate aspect of the present disclosure.

### DETAILED DESCRIPTION OF THE DISCLOSURE

The present disclosure now will be described more fully hereinafter with reference to the accompanying drawings, in which some, but not all aspects of the disclosures are shown. Indeed, these disclosures may be embodied in many different forms and should not be construed as limited to the aspects set forth herein; rather, these aspects are provided so that this disclosure will satisfy applicable legal requirements. Like numbers refer to like elements throughout.

Aspects of the present disclosure are directed to methods of conducting or performing various oral and/or maxillofacial procedures, in relation to a fiducial marker formed by secure and physical interaction between a fiducial device and the patient, while improving access to the surgical site and minimizing or eliminating risk of injury to the patient.

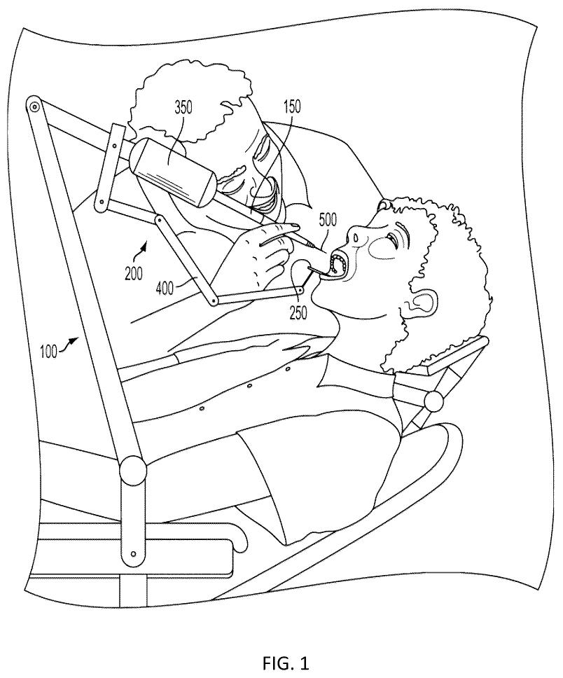

FIG. 1 and FIGS. 2 and 3 illustrate alternate aspects of a system for conducting a guided oral and maxillofacial procedure, according to the present disclosure, the system being generally indicated by the numeral 100. In some oral and maxillofacial procedures an imaging step is generally involved, wherein CT or other appropriate images of a site such as, for example, the patient's jaw/oral/maxillofacial structure are obtained, and any anomalies with the site diagnosed (e.g., whether the patient requires bone grafts to prepare an implant area). The practitioner then corrects any anomalies with the site and proceeds with the oral/maxillofacial procedure based on the conditions associated with the images of the site (e.g., the patient's jaw/tooth structure), once the appropriate incisions (if required) have been made in the site structure (e.g., the patient's gum). In other instances, the site and associated site structure may be non-human/non-anatomical in nature.

A system 100 for conducting a guided object-related procedure, such as a guided oral and maxillofacial procedure, according to various aspects of the present disclosure, addresses certain limitations of current object-related (e.g., oral and/or maxillofacial) procedures by providing a guided procedure-conducting device 150 configured to be guided with respect to the operative portion of the object-related procedure (e.g., to “prepare” the site within the patient's mouth). That is, the procedure-conducting device 150 is operably engaged with a guidance device 200. The guidance device 200 is adapted to be in communication with a site having an object associated therewith (e.g., with the mouth of the patient having teeth in the mouth). For example, the engagement with the site may be through a fiducial device such as a splint 250 or other engaging member. In one instance, the splint 250 is configured to engage the site (e.g., the patient's mouth) or an object associated therewith (e.g., teeth) in a “firm” or secure interaction (e.g., the splint 250 is engaged with the patient's teeth and does not move with respect to the patient's mouth). Since the splint 250 does not move with respect to the site (e.g., the patient's mouth), the disposition of the splint 250 is known, and thus can be configured to provide a fiducial marker (e.g., a known origin or coordinate) which can be used, for instance, to guide the procedure-conducting device to prepare the site, the object associated with the site, or to perform the object-related procedure. In one aspect, the splint 250 is configured to be “universally applicable” (e.g., capable of forming the secure engagement with any site such as the mouth of any patient), or at least applicable across a particular range of sites (e.g., one size fits a certain size or age of patient). In order to determine the fiducial marker, according to one aspect of the disclosure, the splint 250 may be engaged with the site, such as the patient's teeth or jaw, and the patient's jawbone structure then imaged using, for example, CT or any other suitable imaging technique such as, for instance, MRI.

More particularly, one underlying premise of the subject matter of the present application is that the guided oral/maxillofacial system is just one example of a system that accounts for any movement of an object (e.g., the patient's jaw or mouth, or a non-human/non-anatomical object such as a piece of wood) in which an element, object, implant, or crown (e.g., a tooth, or a nail to be implanted in the piece of wood) is to be manipulated with precision. Accordingly, the problem to be solved is how to determine a path that the procedure-conducting device is supposed to travel in order to perform the object-related procedure at the site or in relation to the object associated therewith, and to appropriately adjust that path to reestablish the desired orientation of the interaction by the procedure-conducting device with respect to the object/site, upon movement of that object/site. In this case, the “site” or “object” associated therewith can be anything movable—it can be the mouth of a patient having a wisdom tooth removed, a root canal performed, a crown installed, or an implant implanted, or a block of wood receiving a nail.

One skilled in the art will also appreciate that the splint 250 may be configured in many different manners to accomplish the desired function as discussed herein. For example, the splint 250 may be rigidly attached to the site or the object at the site in an appropriate manner depending on the condition of the site. That is, for example, if the patient has some strong teeth capable of supporting the splint 250, the splint 250 can be attached to the teeth with an adhesive or with a suitable clamp. For edentulous patients (e.g., without teeth), bone pins may be drilled through the splint 250 and into the patient's jawbone structure to fasten the splint 250 securely into place. The splint 250 may also be attached to the jawbone structure of any patient using, for example, appropriate bone screws. In one aspect, the positioning of the splint 250 with respect to site (e.g., the patient's mouth) may not be critical or important, as long as the splint 250 remains rigidly in place. A fiducial marker (not shown) may then be attached to, or otherwise incorporated into, the splint 250, wherein the fiducial marker may be configured to have a geometry or other characteristic or feature that uniquely defines the fiducial marker in a three-dimensional space (e.g., such that the fiducial marker is readily identified in images of the site represented by the patient's jawbone structure). In such instances, the fiducial marker may be comprised of, for example, a radiopaque material that can be clearly defined in the image (e.g., CT or MRI).

In one instance, the procedure-conducting device 150 is engaged with an articulating arm member 350 (e.g., a robotic arm) which determines a range of motion of the procedure-conducting device 150. The guidance device 200, in such instances, may further comprise a communication element 400 and/or a controller device 450 in communication between the splint 250 and the procedure-conducting device 150 and/or the arm member 350. For example, the communication element 400 may comprise a mechanical linkage connecting the splint 250 to the procedure-conducting device 150/arm member 350, via a common base 180. That is, the communication element 400 may comprise, for example, a mechanically-tracked arm which attaches to the splint 250 engaged with the object/site at one end, and to the base 180 at the opposing end. In some instances, the arm may be attached to the splint 250 (rigidly and in a known, repeatable manner) with an attachment mechanism comprising a kinematic mount.

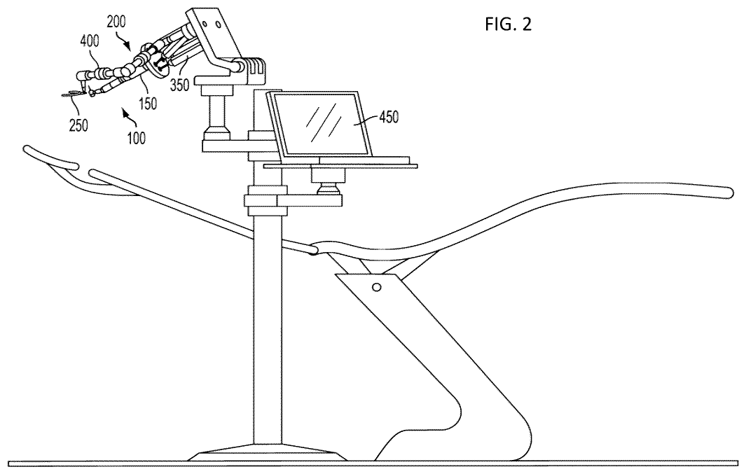

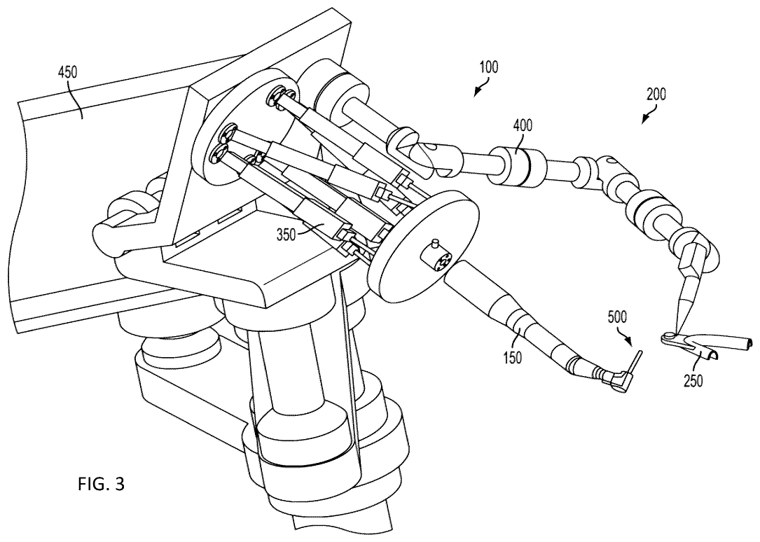

Attached to the site/object in this manner via the attachment mechanism and the splint 250, the communication element 400 provides data (whether constantly, selectively, or otherwise as necessary) about the position of the site/object (e.g., with respect to the fiduciary marker) to the controller device 450 which, in turn uses this data to guide or control the procedure-conducting device 150/arm member 350, while still providing for accurate guidance thereof in the event that the site/object moves (e.g., the procedure-conducting device 150/arm member 350 is controlled and guided by the controller device 450 in reference to the communicated position of the fiduciary marker). However, one skilled in the art will appreciate that the splint 250 and/or the fiducial marker determined thereby may be communicated to the controller device 450 and/or the procedure-conducting device 150/arm member 350 in many different manners. For example, the fiducial marker may be communicated to the controller device 450 and/or the procedure-conducting device 150/arm member 350, via a communication element 400 comprising a wireless transceiver, a hardwire connection, an optical communication system, or any other suitable mechanism, whether electrical, mechanical, electromechanical, or optical in nature. In any instance, the controller device 450 (e.g., a computer device as shown in FIGS. 2 and 3) is configured and arranged for determining data associated with the fiducial marker from the image of site (e.g., the patient's mouth) having the splint 250 disposed therein or engaged therewith, and for appropriately communicating guidance associated with the fiducial marker to the procedure-conducting device 150/arm member 350.

In one aspect, the controller device 450 may be further configured to receive the image of the site (e.g., the patient's jawbone structure having the splint 250 therein). In some instances, the controller device 450 may be further configured to function as a procedure planning device and to be capable of executing a procedure routine that may comprise software, hardware, or a combination thereof. The procedure routine thus allows the practitioner to create, for example, a virtual procedure plan based on the captured image, whether in two dimensions or three dimensions, and to manipulate the image(s) of the site in conjunction with respect to the planned object-related procedure in order to develop the virtual procedure plan or determination of the appropriate procedure for the site/object in conjunction with a computerized model based on the image(s). In some aspects, the procedure routine, virtual procedure plan, and/or procedure determination may be created in relation, for example, to a coordinate system (relative or absolute), as will be appreciated by one skilled in the art, for associating the procedure parameters with the fiducial marker. As such, the virtual procedure plan is in registration with and formed with respect to the fiducial marker. In other aspects, the controller device 450 may include a peripheral device (e.g., a trackball or joystick in conjunction with, for example, 3D goggles, all not shown) to assist with or otherwise permit virtual manipulation of factors associated with the procedure with respect to the image(s) in order to, for example, align the objects such as dental implant(s) relative to each other or relative to the site or adjacent objects such as adjacent teeth, to shape an object such as a tooth for receiving a crown, to perform a root canal, to remove a wisdom tooth, and/or to align the dental implant(s) relative to the jawbone structure. The controller device 450 may be further configured to direct or perform such manipulation manually, automatically, or semi-automatically, as necessary or desired. Because the virtual factor(s) may be manipulated in a similar manner to the image(s), the orientation, direction, or placement of the virtual factor(s) may represent the desired actual placement of the factor with respect to the site (e.g., the patient's jawbone structure), thus providing an intuitive interface for planning the object related (e.g., oral/maxillofacial) procedure.

In aspects where the splint 250/fiducial marker approach is used, the site/object (e.g., patient) is automatically registered with the system 100 once the communication element 400 (arm) is attached to the splint 250 via the kinematic mount of the attachment mechanism, or otherwise entered into communication and registration therewith. For example, the communication element 400 may not be physically engaged with the splint 250/kinematic mount, but may be in communication therewith by wireless communication, by optical communication, or by any other communication system not requiring a physical engagement. That is, the fiducial marker is automatically determined from the image(s) of the site/object (e.g., the patient's jawbone structure), and the alignment and location thereof in physical space is known due to the kinematic mount providing communication between the arm and the splint 250. One skilled in the art will appreciate, however, that other alignment approaches may be implemented that do not necessarily require a fiducial marker. For example, in some instances, a surface matching technique can be implemented. More particularly, the site (e.g., the patient's jawbone structure) may be manipulated into a 3D configuration in the captured image(s). A suitable scanning device (e.g., a physical pointer or other imaging device such as an ultrasound transducer or OCT (optical coherence tomography) scanner may be attached to the end effector of the arm member 350 such that the tip of the arm member 350 is capable of scanning the site (e.g., the patient's jawbone structure) to “surface match” the captured and manipulated image(s) with an actual scan of the site.

One skilled in the art will further appreciate that the association of the fiducial marker with the site (e.g., the patient's anatomy), via the controller device 450, may be accomplished in different manners. For example, with respect to the registration of the image (e.g., CT scan) to the fiducial marker, one method could involve the site (e.g., the jaw structure of the patient) being imaged with the fiducial marker in place, as previously discussed, wherein the patient would then be substantially immediately subjected to the procedure. Such a scheme may be beneficial, for example, in reducing the number of visits to the practitioner by the patient. However, in some instances, the practitioner may not have the imaging capabilities at hand, or may prefer to carefully determine the virtual procedure plan before carrying out the procedure. In both such instances, the patient will likely be required to return to the practitioner at a later time. Accordingly, in such situations, a pre-operative imaging procedure (e.g., CT scan) may be performed on the jaw structure of the patient, without a fiducial marker in place (e.g., a “normal” scan by which the practitioner can determine the virtual procedure plan). This pre-operative imaging procedure can thus be performed, for example, at the practitioner's site, or at a dedicated scanning/imaging center. Subsequently, immediately prior to the object-related (e.g., oral/maxillofacial) procedure being performed, and with the fiducial marker(s) engaged with the site (e.g., the jaw structure of the patient), the practitioner may capture another image (e.g., CT scan, panoramic x-ray, or two single x-rays) of the site. The controller device 450 may thus also be configured to correlate the pre-operative image (used to determine the virtual procedure) with the “day of” image so as to register the fiducial marker(s) with respect to the original pre-operative image. Such a registration or correlation procedure may be implemented in hardware, software, or a combination thereof, as will be appreciated by one skilled in the art. The object-related procedure could then proceed as otherwise disclosed herein.

In any instance, the communication element 400 is configured to communicate via the controller device 450 with the arm member 350 in a manner known to the system 100, such that the position/movement characteristics of the end effector of the arm member 350 are also known. This communication between the communication element 400 and the arm member 350 thus allows the procedure-conducting device 150 to be registered with respect to the fiducial marker (or other reference with respect to the site/object) attached to the site/object via the splint 250, the kinematic mount, the communication element 400, the controller device 450, and the arm member 350. In this manner, the virtual procedure plan, planned through the controller device 450, may be accomplished in relation to the fiducial marker (or other reference with respect to the patient) and thus translated or otherwise communicated to the system 100 for directing the procedure-conducting device 150.

The procedure-conducting device 150 is disposed in or otherwise engaged with the end effector of the arm member 350 (robotic arm). The arm member 350 may be configured, for example, to provide six degrees of freedom and can also be configured to restrict or otherwise control the movement of the procedure-conducting device 150. Further, in one example, the arm member 350 may have a miniature parallel structure to which the procedure-conducting device 150 is secured and allowed to have full freedom of movement when not in the mode of conducting a procedure. Since the procedure-conducting device 150 is attached to the end effector of the arm member 350, the site/object interacting portion (e.g., the cutting/drilling/abrading/ablating tip) 500 (see, e.g., FIGS. 1 and 3) of the procedure-conducting device 150 must be in a known position (e.g., known to the system 100) relative to the arm member 350. In some aspects, in order to calibrate the site/object interacting portion 500 of the procedure-conducting device 150 with respect to the fiducial marker, a calibration element may be engaged with the procedure-conducting device 150 via a kinematic coupling (e.g., rigidly mounted thereto in a known, repeatable manner). One skilled in the art will thus appreciate that the site/object interacting portion 500 of the procedure-conducting device 150 can then be calibrated with various tip calibrating methods (e.g., invariant point, etc.). Once calibrated, the calibration element is replaced with a cutting/drilling/abrading/ablating element in the procedure-conducting device 150, in a known and repeatable manner, so that the calibration parameters (e.g., the position of the distal-most point and axis of cutting/drilling) associated with the site/object interacting portion 500 are maintained as calibrated.

In one aspect of the disclosure, as discussed herein, the system 100 may be configured such that the site/object interacting portion 500 or the procedure-conducting device 150 comprises a plurality of interchangeable procedural devices. For example, in one instance, the procedural device is an object-removal device (e.g., a tooth removal device), and the object-related procedure is an object-removal procedure (e.g., a tooth removal procedure). More particularly, in such an instance, the object-removal device is an ablation device, and the object-related procedure is a wisdom tooth removal procedure. In another aspect, the procedural device is a drilling device or an abrading device, and the object-related procedure is a root canal procedure. In yet another aspect, the procedural device is an abrading device, and the object-related procedure is preparing an object such as a tooth for receiving a crown.

With the alignment with respect to the site/object (e.g., patient) established and known by the system 100, and the virtual procedure plan developed through the controller device 450, the procedure (e.g., cutting/drilling/implanting/abrading/ablating) can then be initiated by the practitioner moving the procedure-conducting device 150 toward the site (the patient's mouth having the splint 250 engaged therewith during the procedure). In such instances, the controller device 450 is configured to control the movement of the procedure-conducting device 150 via the arm member 350 such that the action of the practitioner merely moves the site/object interacting portion 500 (e.g., the cutting/drilling/abrading/ablating element) to the appropriate starting position for the procedure, with respect to the site, such as the patient's jawbone structure, as determined by the controller device 450 and dictated by the virtual procedure plan. Once the cutting/drilling/abrading/ablating element is in the position dictated by the controller device 450, the operative portion of the procedure can then be initiated, wherein the controller device 450 may further dictate other parameters of the procedure-conducting device 150 such as, for example, the orientation of the manipulation path of the cutting/drilling/abrading/ablating element and the cutting/drilling/abrading/ablating distance along that path from an origin, also according to the virtual procedure plan.

In these instances, one distinction of the system 100 disclosed herein is that the procedure-conducting device 150 is not guided by the practitioner, but is only urged by the practitioner along a procedural route determined via the virtual procedure plan and implemented via the controller device 450 and the arm member 350. That is, the system 100 may be configured to restrict the practitioner to performing the procedure with respect to the site, as determined via the virtual procedure plan and implemented via the controller device 450 and the arm member 350, whereby the controller device 450 controls the allowable movement of the arm member 350 (and thus the procedure-conducting device 150) in accordance with the virtual procedure plan created from the image(s) of the site/site structure, such as the patient's jawbone structure. That is, the controller device 450/guidance device 200 is configured to physically regulate movement of the procedure-conducting device 150. For instance, the system 100 may be configured for restricted movement of the arm member 350/procedure-conducting device 150, as communicated to the practitioner through tactile/haptic feedback, where, for example, the arm member 350/procedure-conducting device 150 may be easier to move according to the virtual procedure plan, and more difficult to move if deviating from the virtual procedure plan. One skilled in the art will also appreciate, however, that the physical structure of the arm member 350/procedure-conducting device 150 to provide fully controlled movement according to the virtual procedure plan (e.g., due to vibration, flexing of components, and/or excessive force applied by the practitioner) and, as such, the system 100 may be further configured to provide other manners of tactile/haptic feedback to the practitioner such as, for example, via a deviation warning indicia or any other suitable audio and/or visual mechanism. Therefore, the system 100 includes provisions for actually implementing the virtual procedure plan, and thus facilitates a more accurate procedure, rather than merely warning the practitioner if any procedural parameters may be inaccurate. One skilled in the art will also appreciate, however, that, in some instances, the system 100 may be further configured to autonomously accomplish the virtual procedure plan, without the manipulation of the practitioner, through automatic manipulation of the arm member 350/procedure-conducting device 150 via the controller device 450.

In one exemplary surgical procedure using a system 100 for conducting a guided oral and maxillofacial procedure, as disclosed herein, the splint 250 (e.g., mouthpiece) is first attached to the patient's teeth, and thus provides a fiducial marker. The patient's jawbone structure is then imaged (with the splint 250 in place and engaged with the patient's teeth) using, for example, CT or any other appropriate imaging technique (e.g., MRI), and the image(s) communicated with the controller device 450. The controller device 450 may be further configured to be capable of executing a procedure routine, thus allowing the practitioner to develop a procedure plan for the patient, for example, by manipulating a virtual procedure-conducting device 150 with respect to a wisdom tooth in the captured image(s). Once the virtual procedure plan is created, the communication element 400 is engaged with the splint 250 (attached to the patient's mouth, with the patient being positioned in a suitable position to initiate the procedure, or actuation of the non-physical communication therebetween). The arm member 350, procedure-conducting device 150, and interacting portion 500 thereof, are then calibrated by the practitioner (or automatically by the controller device 450), before the actual cutting/drilling/abrading/ablating element of the procedure-conducting device 150 is used by the practitioner (or autonomously via the controller device 450), via the procedure-conducting device 150 as guided by the arm member 350 and the controller device 450, to accomplish the procedure as planned and dictated by the virtual procedure plan. Since the splint 250 remains attached to the patient during calibration and during the procedure, the splint 250/fiducial marker also remains in direct and continuing communication with the guidance device 200/controller device 450 during the procedure. As such, the arm member 350, procedure-conducting device 150, and interacting portion 500 thereof are manipulated during the procedure in direct relation with the splint 250/fiducial marker.

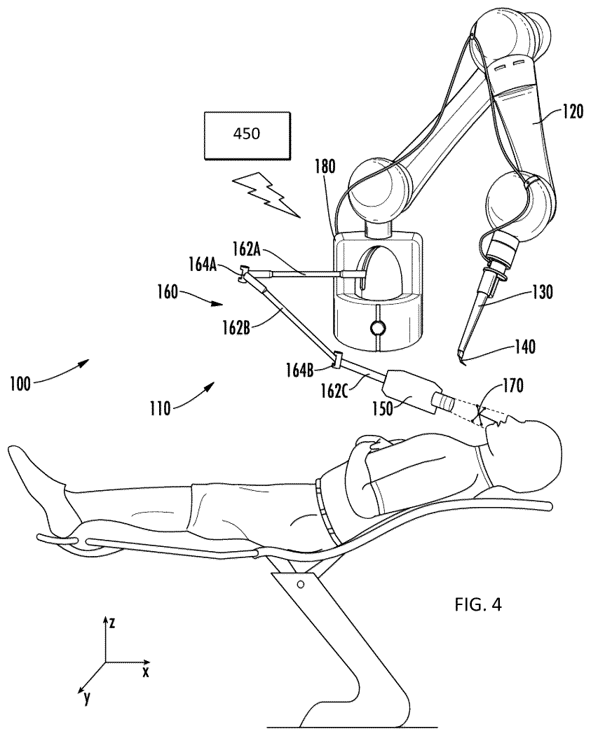

FIG. 4 illustrates a surgical robot system 100, according to an alternate aspect of the present disclosure, having a tracking and guidance arrangement 110 for tracking site/object motion during robotic surgery, wherein the tracking aspect is not physically in communication with the site/object. The tracking and guidance arrangement 110 and/or the surgical robot system 100 may be configured for and readily applicable or adaptable to various surgical procedures (e.g., any procedure associated with a site/object, such as skull surgery, cars, nose, and throat (ENT) surgery, orthopedic surgery, or any other surgical procedure associated with an anatomy of a patient). In particular aspects, the tracking and guidance arrangement 110 comprises, for example, a hybrid (e.g., combined) mechanical and optical tracking and guidance arrangement (see, e.g., FIG. 4), or a hybrid mechanical and electromagnetic tracking and guidance arrangement, each of which provides increased freedom of movement, minimized line-of-sight requirements, reduced interference potential, etc. One skilled in the art will appreciate, however, that other technology combinations for a hybrid tracking and guidance arrangement 110 are also contemplated.

Generally, and in reference to FIG. 4, the tracking and guidance arrangement 110 comprises a site/object interacting device 130, including a guide arm 120, such as, for example, an articulating arm member (e.g., a robotic arm), and an instrument 140 (e.g., a surgical instrument). The instrument 140 is configured to engage a distal end of the guide arm 120, and is adapted to interact or otherwise communicate with a site/object, such as a maxillofacial anatomy (e.g., a jaw or mouth) of the patient, while being guided by the guide arm 120. In some aspects, the site/object interacting device 130 may be referred to herein as a “cutting device”, “drilling device”, “abrading device,” “ablating device,” “site preparation device”, “procedure-conducting device”, or the like, and this reference is intended to indicate the particular instrument 140 engaged with the guide arm 120. As such, the site/object interacting device 130 and the instrument 140 may be interchangeably referred to herein as being configured for a particular corresponding purpose or procedure, with the understanding that such reference is intended to indicate that the instrument 140 element of the site/object interacting device 130 is configured to be directed or guided, via the guide arm 120, with respect to an invasive portion, or at least a site/object interacting portion of a robotic surgery procedure (e.g., to “prepare” the site/object, or otherwise interact with the jaw or mouth of the patient). As also previously disclosed, various instruments 140 may be configured so as to be interchangeably received by the guide arm 120/site/object interacting device 130. Thus, the tracking and guidance arrangement 110 may be readily adapted for various surgical procedures that would benefit from the guidance provided by the disclosed aspects of the system 100.

In some aspects, one or more actuators (not shown) may be engaged with the guide arm 120 and may be configured and arranged to cooperate to guide (e.g., translate in a particular direction (horizontal and/or vertical), and/or rotate about an axis) the distal end of the guide arm 120 in six degrees of freedom upon manipulation by the user to accomplish the surgical procedure. The guide arm 120 can also be configured to restrict or otherwise control the movement of the site/object interacting device 130, and thus the instrument 140. Further, in some instances, the guide arm 120 may have a miniature parallel structure to which the instrument 140 is secured and allowed to have full freedom of movement. Since the instrument 140 comprises or is attached to the distal portion of the guide arm 120, the site/object interacting portion (e.g., the cutting/drilling tip) is the instrument 140 of the site/object interacting device 130, and the instrument 140 thus must be in a known spatial position (e.g., known to the system 100 relative to the guide arm 120).

In some aspects, the instrument 140 is guided or directed, via the guide arm 120, according to spatial relations as determined by the tracking and guidance arrangement 110. In this manner, the tracking and guidance arrangement 110 also comprises a detector 150 connected to a distal end of an articulating arm 160 and co-operable therewith, and a fiducial marker 170 coupled to the site/object, such as the jaw or mouth of the patient. The detector 150 can comprise an optical detector (e.g., camera) or an electromagnetic detector (e.g., electromagnetic emitter) configured to interact with the fiducial marker 170, as well as other types of detectors (e.g., an acoustic detector) configured to interact with an appropriately-configured fiducial marker 170. The fiducial marker 170 may be a splint or other engaging member configured to couple to a site/object such as a maxillofacial anatomy (e.g., jaw, mouth) of the patient. That is, in one instance, the fiducial marker 170 is configured to engage the site/object (e.g., the patient's mouth or jaw) in a “firm” or secure interaction (e.g., a splint is engaged with the patient's teeth and does not move with respect to the patient's mouth). In this instance, since the splint does not move with respect to the site/object, an initial spatial position of the splint in a relative coordinate system or three-dimensional space (e.g., an X, Y, Z system) may be determined. Thus, the splint can be configured to provide a fiducial marker (e.g., a known origin or coordinate element formed by the secure interaction with or otherwise associated with or attached to the splint), which can be used, for instance, to guide the instrument 140 of the site/object interacting device 130, via the guide arm 120, during the robotic surgery.

In some aspects, the site/object interacting portion/instrument 140 of the site/object interacting device 130 may be registered or calibrated with respect to the fiducial marker 170. For example, a calibration element (not shown) may be engaged with the site/object interacting device 130 via a kinematic coupling (e.g., rigidly mounted thereto in a known, repeatable manner). One skilled in the art will thus appreciate that the site/object interacting portion/instrument 140 of the site/object interacting device 130 can then be calibrated with various tip calibrating methods (e.g., invariant point, etc.). Once registered, the calibration element may be replaced with a cutting/drilling element (instrument 140) in the site/object interacting device 130, in a known and repeatable manner, so that calibration parameters (e.g., a position of a distal-most point and axis associated with the interacting portion/instrument 140) are maintained as registered.

In one aspect, the fiducial marker 170 is configured to be “universally applicable” (e.g., capable of forming the secure engagement with a site/object such as the anatomy of any patient), or at least applicable across a particular range of sites/objects (e.g., one size fits a certain size or age of patient). In order to determine a reference origin associated with the fiducial marker 170, according to one aspect of the disclosure, the fiducial marker 170 (e.g., a splint or other engaging member) may be engaged with an object such as the patient's teeth, and the site such as the patient's jawbone structure then imaged using, for example, computerized tomography (CT) or any other suitable imaging technique such as, for instance, magnetic resonance imaging (MRI). In such instances, the fiducial marker 170 may be comprised of, for example, a radiopaque material that can be clearly defined in the image obtained, e.g., by CT or MRI, such that the fiducial marker 170 is readily identifiable, or is otherwise detectable, in images of the site such as the patient's jawbone structure. The fiducial marker 170 can thus be established, for instance, as a reference origin of a relative coordinate system or three-dimensional space.

One skilled in the art will appreciate that the fiducial marker 170 may be configured in many different manners to accomplish the desired function as discussed herein. In one aspect, the fiducial marker 170 may be configured based on a type of detector 150 implemented in the tracking and guidance arrangement 110. Where the detector 150 is an optical detector, for example, the fiducial marker 170 may comprise reflective markers (e.g., a geometry or other characteristic or feature that uniquely defines the fiducial marker 170 in a three-dimensional space such that the fiducial marker is readily identified in images of the site, or is otherwise detectable and trackable) for the optical detector 150 to track or otherwise interact with (see, e.g., FIG. 4). In another example, where the detector 150 is an electromagnetic detector, the fiducial marker 170 may comprise an appropriate sensor or emitter for the electromagnetic detector 150 to track or otherwise interact with.

Accordingly, in some aspects of the present disclosure, the detector 150 may be configured to or be capable of being positioned adjacent to the fiducial marker 170, via the articulating arm 160, in order to track movement of the site/object by near proximity interaction with the fiducial marker 170. Notably, the tracking and guidance arrangement 110 illustrated in FIG. 4 is not configured such that the detector 150 and the fiducial marker 170 are physically connected. Rather, the articulating arm 160 is advantageously configured to position the detector 150 adjacent or near the fiducial marker 170. For example, the articulating arm 160 is configured to position the detector 150 within several centimeters of the fiducial marker 170. In this manner, a site/object is not physically tethered to the surgical robot system, and the detector 150 may be positioned in a range suitable to interact with the fiducial marker 170, without some of the limitations encountered in the prior art such as, for example, impedance of communication (e.g., interruption of the line of sight in the case of an optical detector), interference, or distance of the detector from the fiducial marker.

The articulating arm 160 may comprise a plurality of serially-disposed sections 162A-C, with adjacent sections 162A-C being connected by a joint 164A-B. The joints 164A-B may be kinematic mechanisms that enable each of the serially-disposed sections 162A-C to be independently positionable (e.g., translatable, movable, rotatable) within the relative coordinate system or three-dimensional space. In FIG. 4, three serially disposed sections 162A-C are illustrated with a first section 162A having a proximal end mounted to a base 180, a second section 162B connected at a proximal end to a distal end of the first section 162A by a first joint 164A, and a third section 162C connected at a proximal end to a distal end of the second section 162B by a second joint 164B. The detector 150 is connected to a distal end of the third section 162C using, for instance, a mechanical linkage. For example, an additional joint similar to joints 164A-B may be disposed at the distal end of the third section 162C and/or at the proximal end of the first section 162A at which the articulating arm 160 is mounted or otherwise coupled to the base 180. Otherwise, the detector 150 may be rigidly connected to the distal end of the third section 162C. In this manner, manipulation of one or more of the serially-disposed sections 162A-C of the articulating arm 160 may enable the detector 150 to pivot, move, and/or otherwise be positioned in a desired position relative to the fiducial marker 170. As one of ordinary skill in the art will note, a number of serially disposed sections and/or joints more or less than the number illustrated in FIG. 4 may be utilized in the articulating arm 160.

In some aspects, the articulating arm 160 is mounted to the base 180 such that the articulating arm 160 and the guide arm 120 are operably connected, coupled, or in communication via the base 180. For example, the articulating arm 160 and the guide arm 120 may be mechanically linked to one another at proximal ends, at the base 180, or at another location along a length of each of the arms. In other aspects, the articulating arm 160 may be mounted to the base 180 such that the articulating arm 160 and the guide arm 120 are disposed in a spaced-apart relation relative to one another. Regardless, the base 180 may be, advantageously, mobile for ease of use in a variety of different spaces, patient positions (e.g., supine, upright, reclined), surgical needs, etc. Otherwise, the articulating arm 160 and/or the guide arm 120 may be mounted to a non-mobile base (e.g., a stationary platform, such as a wall, ceiling, floor, etc.). Whichever the manner in which the articulating arm 160 and/or the guide arm 120 are mounted, the resulting mounting arrangement may enable the articulating arm 160 to position the detector 150 adjacent to the fiducial marker 170, and may allow the guide arm 120 of the site/object interacting device 130 to direct the instrument 140 to interact with the site/object, such as the maxillofacial anatomy of the patient.

As FIG. 4 discloses a tracking and guidance arrangement 110 where the detector 150 and the fiducial marker 170 are disposed adjacent to one another rather than coupled together, a spatial relation between the fiducial marker 170 and the detector 150 may be determined based on data (e.g., tracking data) resulting from the interaction between the fiducial marker 170 and the detector 150. In order determine the spatial relation between these two components, as well as perform other functionality associated with tracking and guidance for a robot surgical system 100, the tracking and guidance arrangement 110 may further comprise a controller device 450 including a hardware processor and memory operably engaged with one or more components of the tracking and guidance arrangement 110. As illustrated in FIG. 4, for example, the controller device 450 is in wireless communication via a communication element (not shown) with at least the detector 150, the articulating arm 160, the guide arm 120, the site/object interacting device 130, and the instrument 140. In some aspects, the communication element may be a wireless transceiver, a hardwire connection, or any other suitable mechanism, whether electrical, mechanical, electromechanical, acoustic, or optical in nature.

The controller device 450 may comprise a special purpose computer device disposed either separately from or integrated with the base 180. The controller device 450 may be configured to determine a reference point or origin associated with the fiducial marker 170 in a defined relative coordinate system or three-dimensional space, to articulate the detector 150 relative to the fiducial marker 170 so that the detector 150 is disposed in a desired position adjacent to the fiducial marker 170, to determine a spatial position of the detector 150 in the defined relative coordinate system or three-dimensional space once the detector 150 is articulated into the desired position, to initiate interaction between the detector 150 and the fiducial marker 170, to receive data from the detector 150 relative to the interaction thereof with the fiducial marker 170, and to determine a spatial relation between the fiducial marker 170 and the detector 150 based on the data.

In some aspects, determining a reference point or origin associated with the fiducial marker 170 may be accomplished by imaging the fiducial marker 170 coupled to the site/object while the site/object is in an initial position in a defined relative coordinate system or three-dimensional space. The controller device 450 may be configured to initiate the imaging by interfacing with whatever imaging modality is utilized (e.g., CT or MRI imaging). The image(s) or data may be stored in a data storage device (not shown) associated with the controller device 450 and utilized to establish an initial position of the fiducial marker 170 within the relative coordinate system or three-dimensional space as being an origin.

In some aspects, articulating the detector 150 relative to the fiducial marker 170 so that the detector 150 is disposed in a desired position adjacent to the fiducial marker 170, may be accomplished by manipulating one or more of the serially-disposed sections 162A-C relative to the fiducial marker 170. For example, a peripheral device (e.g., a trackball or joystick in conjunction with, for example, 3D goggles, all not shown) associated with the controller device 450 may be used to assist with or otherwise permit virtual manipulation of one or more of the serially-disposed sections 162A-C of the articulating arm 160. Otherwise, an operator of the robot surgical system 100 may manually manipulate one or more of the serially-disposed sections 162A-C of the articulating arm 160 to move the detector 150 into the desired position.

In some aspects, a spatial position of the detector 150 in the defined relative coordinate system or three-dimensional space, once the detector 150 is articulated into the desired position, may be determined by the controller device 450 receiving angular relations communications from one or more position-indicating device (e.g., an encoder). More particularly, the one or more position-indicating devices (not shown) may be engaged with one or more of the joints 164A-B for indicating an angular relation between the serially-disposed sections 162A-C engaged therewith in the defined relative coordinate system or three-dimensional space. The position-indicating device and the controller device 450 may be in communication with one another such that the one or more position-indicating devices communicate to the controller device 450 the angular relations of the joints within the defined relative coordinate system or three-dimensional space. Where the detector 150 is disposed at a distal end of the third section 162C, the controller device 450 may be configured to determine the spatial position of the detector 150 based on the angular relations of each joint 164A-B communicated thereto, as well as based on other information, such as, for example, a length of each section 162A-C. Such data relating to the spatial position of the detector 150 may be stored in a data storage device associated with the controller device 450.

In some aspects, once the articulating arm 160 is in a desired position in the defined relative coordinate system or three-dimensional space, the controller device 450 may be configured to initiate interaction between the detector 150 and the fiducial marker 170. The controller device 450 may be in communication with the detector 150 and may be configured to initiate and/or actuate operation of the detector 150. For example, where the detector 150 is a camera or other image capturing device, the controller device 450 may be configured to actuate the detector 150 to acquire images of the fiducial marker 170 coupled to the site/object at a specified frame rate. In such aspects, the peripheral device associated with the controller device 450 may be configured to continuously assist or otherwise permit virtual manipulation of the one or more serially disposed sections 162A-C of the articulating arm 160 so that optimal spacing (e.g., several centimeters) is maintained between the detector 150 and the fiducial 170. In other such aspects, feedback communication between the detector 150 and the controller device 450 with regard to spacing between the detector 150 and the fiducial marker 170 may be configured to automatically assist or otherwise permit virtual manipulation of the one or more serially disposed sections 162A-C of the articulating arm 160 so that optimal spacing is maintained between the detector 150 and the fiducial 170.

In some aspects, the data acquired from the detector 150 may be transmitted to the controller device 450, such that the controller device receives the data from the detector 150 relative to the interaction thereof with the fiducial marker 170. The detector 150 and the controller device 450 may be in either wired or wireless communication via the communication element.

In some aspects, to determine a spatial relation between the fiducial marker 170 and the detector 150, the controller device 450 may be configured to utilize the reference point or origin associated with the fiducial marker 170 and the spatial position of the detector 150 in the desired position to determine a first spatial relation therebetween. Subsequently, the controller device 450 may be configured to utilize the images acquired from the detector 150 to track movement of the fiducial marker 170 in the defined relative coordinate system or three-dimensional space. For example, the controller device 450 may be configured to compare the data regarding the original reference point or origin associated with the fiducial marker 170 against subsequent data acquired by the detector 150 in order to determine if a spatial position of the fiducial marker 170 has changed. Using this comparison in light of the known spatial position of the detector 150, the controller device 450 may determine a changed spatial relation between the fiducial marker 170 and the detector 150. In this manner, movement of the site/object may be continuously tracked by the detector 150.

Otherwise, as previously disclosed, aspects of the surgical robot system 100 or the controller device 450 may also comprise a planning device or otherwise include planning functionality for allowing a user to develop a virtual procedure plan, in conjunction with the hardware and/or software of the system 100. In some aspects, the virtual procedure plan may be created in relation, for example, to the defined relative coordinate system or three-dimensional space (relative or absolute), as will be appreciated by one skilled in the art, and configured to associate planning parameters with the fiducial marker 170 (or other reference with respect to the site/object). The controller device 450 may be configured to register the site/object interacting device 130 and/or the instrument 140 with the fiducial marker 170. In some aspects, the planning parameters may define a spatial relation directly between the fiducial marker 170 (still affixed to the patient) and the site/object interacting device 130 at different portions of or continuously during the surgical procedure. However, if the site/object moves, the site/object interacting device 130 may need to compensate for site/object movement by returning the instrument 140 to a defined spatial relation between the site/object interacting device 130/instrument 140 and the fiducial marker 170 as defined at a specific point in the virtual procedure plan. In some aspects, an operator of the surgical robot system 100 may perform surgery without the assistance of a virtual procedure plan.

As disclosed, aspects of the present disclosure contemplate that the site/object interacting portion 500 or the procedure-conducting device 150, or the instrument 140 or the site/object interacting device 130, may be interchangeable between various special purpose instruments, such that different surgical procedures can be readily performed with the same system 100, with minimal time and complexity required to suitably adapt the system 100. Such different procedures include, but are not limited to, for example, a wisdom tooth removal procedure, a root canal procedure, or a procedure for preparing a tooth for receiving a crown.

Regardless of the procedure, the method of conducting the procedure utilizing aspects of the system 100 disclosed herein include, for example, forming a secure and physical interaction between a fiducial device and a site/object (e.g., teeth within a mouth of a patient), wherein the secured fiducial device forming a fiducial marker. Further, a virtual procedure (e.g., tooth removal, root canal, tooth preparation for receiving a crown) plan is formed detailing the procedure with respect to the relevant site/object, such as the maxillofacial anatomy of the patient. In any instance, the virtual procedure plan is in registration with and formed with respect to the fiducial marker. Movement of a procedure-conducting device is physically regulated with a guidance device operably engaged with the procedure-conducting device, in response to a controller device in communication with the guidance device and with respect to the fiducial marker. The guidance device is configured to physically regulate movement of the procedure-conducting device, in accordance with the virtual procedure plan and in correspondence with physical manipulation of the procedure-conducting device by the user, to perform the procedure. During the procedure, tactile or haptic feedback is provided to the user, via the procedure-conducting device, if the physical manipulation of the procedure-conducting device by the user causes the procedure-conducting device to deviate from the virtual procedure plan.

Such a method may further comprise imaging the targeted subject with respect to the fiducial marker to facilitate registration of the virtual procedure plan with the fiducial marker. Further, the step of providing tactile/haptic feedback may further comprise allowing movement of the procedure-conducting device in accordance with the virtual procedure plan and physically preventing movement of the procedure-conducting device deviating from the virtual procedure plan. In some instances, providing tactile feedback further comprises vibrating the procedure-conducting device if movement of the procedure-conducting device deviates from the virtual procedure plan.

As otherwise disclosed herein, in some instances, the guidance device in communication with the fiducial device is also physically related therewith. However, in other instances, the guidance device can be engaged in physical or nonphysical communication with the fiducial device.

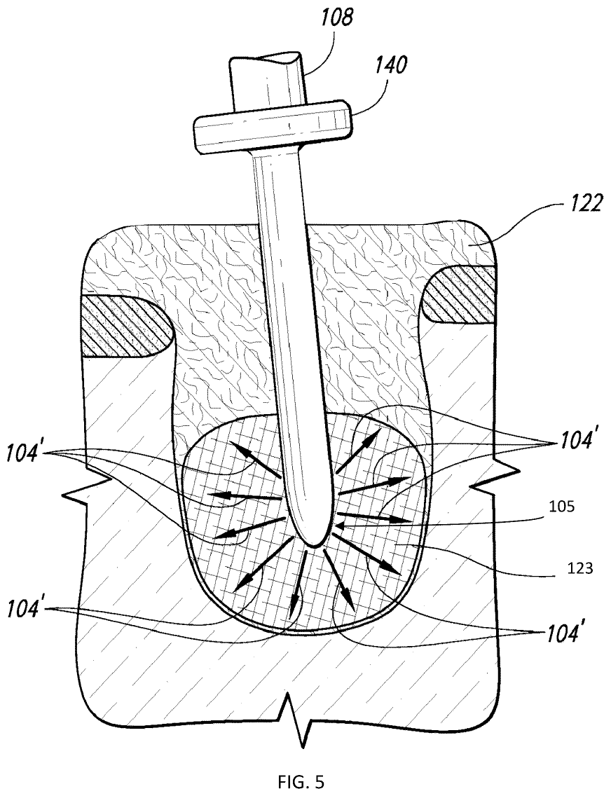

FIG. 5 schematically illustrates one example of the system 100 suitably adapted for an object (e.g., wisdom tooth) removal procedure, particularly where an ablation probe tip 108 has structure suitable for connecting it to the procedure-conducting device 150/site/object interacting device 130. Ablation energy 104′ (e.g., microwave energy, RF energy, irreversible electroporation, cryoablation, ultra-high intensity ultrasound, laser, chemical, thermal or hot tip (e.g. a tip having any source of heat including, but not limited to a light bulb, a soldering iron, or steam heat), and/or mechanical energy) flows from the generator 104 through the ablation probe tip 108 and out to a center of ablation 105 (the focal point of the ablation). The ablation probe tip 108 is insertable through the gingival tissue 122, and into the middle of the tooth bud 123. The center of ablation 105 is at the insertion end of the ablation probe tip 108 such that when the insertion end of the ablation probe tip 108 is positioned at a pre-defined angle (+) and a pre-defined depth (x) during the procedure, the center of ablation 105 substantially coincides with or overlaps the middle of the tooth bud 123. In some aspects, the ablation probe tip 108 includes a mechanical stop structure 140 (e.g. a band, protrusion, or shoulder) for physically limiting the depth of the ablation probe tip 108 into the tooth bud 123, though with the guidance provided by the system 100, the mechanical stop structure may not be necessary. The ablation probe tips 108 may be sharp enough and/or may be strong enough to so that the ablation probe tips 108 can be “self-introducing” or readily pushed through the gingival tissue 122. One skilled in the art will also appreciate that the ablation probe tip 108, or otherwise the procedure-conducting device 150/site/object interacting device 130, may include other devices capable of accomplishing the removal of the object such as the tooth or tooth bud. For example, a drilling device or abrading device, as otherwise disclosed herein, could be implemented in place of or in addition to the ablation probe tip for removal of the object such as the wisdom tooth or wisdom tooth bud. The system 100, in this aspect, is thus configured and arranged to physically regulate movement of the ablation device (the ablation probe tip 108). Moreover, in some aspects, the system 100 is configured to physically regulate movement of the object removal device (e.g., the ablation device) via the guidance device comprising an arm member physically engaged with the object removal device, wherein the guidance device is responsive to the controller device to guide the physical manipulation of the object removal device by the user according to the virtual procedure (e.g., wisdom tooth removal or ablation) plan.

As disclosed herein, any reference to a wisdom tooth removal, or removal of a wisdom tooth, also includes wisdom tooth growth prevention. That is, if the wisdom tooth is already formed, wisdom tooth removal involves removing the formed wisdom tooth. However, if the item of interest is a tooth bud, the “removal” procedure (e.g., ablation of the tooth bud) would actually be a wisdom tooth growth prevention procedure since the tooth bud is ablated and removed prior to the growth of the wisdom tooth. Accordingly, “wisdom tooth removal” as referred to herein will be understood to apply to wisdom tooth removal as well as wisdom tooth prevention.

Further, although the wisdom tooth removal procedure disclosed and claimed herein refers to and recites a tooth removal device (e.g., an ablation device) for removing or preventing growth of the wisdom tooth, one skilled in the art will also appreciate that the wisdom tooth removal procedure may also comprise and use a device for penetrating the soft tissue (e.g., gum tissue or gingival tissue) overlying the wisdom tooth or wisdom tooth bud, prior to the tooth removal device interacting with the wisdom tooth or wisdom tooth bud in the tooth removal procedure. For example, in one aspect, the penetrating device may be combined with the tooth removal device in the form of a “self-introducing” probe. That is, the single probe may have a first portion configured to penetrate the soft tissue, and then a second portion for conducting the tooth removal procedure. In another example aspect, the penetrating device may comprise a drilling device for drilling an access port through the soft tissue to prepare access to the wisdom tooth or wisdom tooth bud for the tooth removal device (e.g. separate devices).

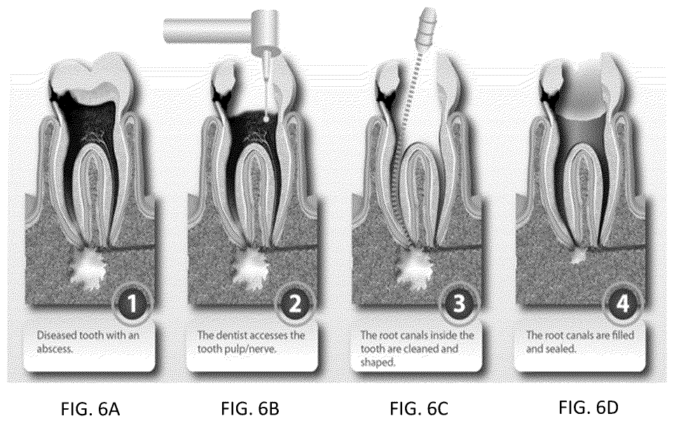

FIGS. 6A-D schematically illustrate an object-related procedure, such as a root canal procedure. When such a procedure is required, a cavity has penetrated into a tooth such that the pulp/nerve within the tooth becomes infected and causes pain to the patient (see, e.g., FIG. 6A). In such instances, the root canal procedure involves drilling a hole in a tooth with a drilling device to access the pulp/nerve within the roots of the tooth (see, e.g., FIG. 6B). Once accessed, the pulp/nerve within the root is filed out or abraded out of the roots by an abrading device (see, e.g., FIG. 6C). The hollowed tooth is then filled with a filler material so that the tooth can remain intact without any nerve pain (see, e.g., FIG. 6D). The system 100, in this aspect, is thus configured and arranged to physically regulate movement of the particular instrument or site/object interacting device (e.g., the drilling device or the abrading device). Moreover, in some aspects, the system 100 is configured to physically regulate movement of the particular instrument or site/object interacting device (e.g., the drilling device or abrading device) via the guidance device comprising an arm member physically engaged with the particular instrument or site/object interacting device, wherein the guidance device is responsive to the controller device to guide the physical manipulation of the particular instrument or site/object interacting device by the user according to the virtual procedure (root canal) plan.

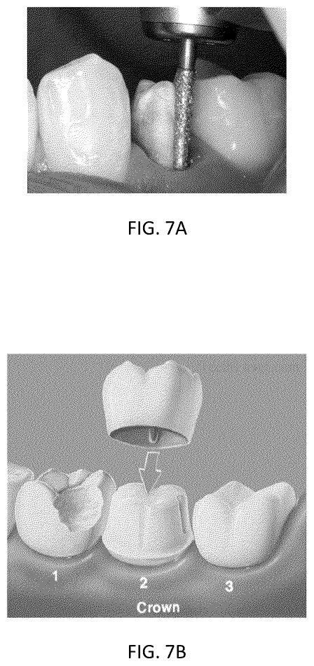

FIGS. 7A and 7B schematically illustrate a procedure involving preparation of an object (e.g., a tooth) for crown placement. That is, when a cavity in a tooth is sufficiently advanced, a dentist may remove a portion of the tooth (see, e.g., FIG. 7A) with an object preparation device such as an abrading device, and replace the removed portion with a porcelain (or zirconium, etc.) crown (see, e.g., FIG. 7B). The system 100, in this aspect, is thus configured and arranged to physically regulate movement of the particular instrument or site/object interacting device (e.g., the object preparation device such as the abrading device). Moreover, in some aspects, the system 100 is configured to physically regulate movement of the particular instrument or site/object interacting device (e.g., the object preparation device such as the abrading device) via the guidance device comprising an arm member physically engaged with the particular instrument, site/object interacting device, or object preparation device, wherein the guidance device is responsive to the controller device to guide the physical manipulation of the particular instrument, site/object interacting device, or object preparation device by the user and according to the virtual procedure (preparation of a tooth for crown placement) plan.

In some aspects, the system 100/controller device 450 can be used in the process for preparation of an object such as a tooth for crown placement to plan the removal of the object material or tooth material (virtual procedure plan) with a custom 3D shape (e.g., virtual sculpting of the abraded tooth). The system 100 is then used to guide (or autonomously or with a dead-man switch) the object preparation device such as the abrading device to remove the specified material from object such as the tooth such that the post-procedure object (tooth) is sculpted in a manner corresponding to the virtual procedure plan. In some aspects, the system 100 (or object preparation device thereof) can also be used to mill or otherwise form a custom shape of the crown from a block of zirconium (or porcelain etc.), including a corresponding receptacle, so that the receptacle in the crown matches the sculpted shape of the tooth remaining after material removal by the abrading device. If necessary or desired, an intra-oral scan may, in some instances, be used as the pre-operative imaging for preparation of the virtual procedure plan. Many modifications and other aspects of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

What is claimed is:

1. A method for conducting a guided oral or maxillofacial procedure, comprising:
securing a fiducial marker included with a fiducial device in a secure and physical interaction with an oral or maxillofacial site having an oral or maxillofacial object associated therewith;
calibrating a calibration element in an oral or maxillofacial procedure-conducting device, the calibration element being interchangeable with a plurality of oral or maxillofacial procedural devices in the oral or maxillofacial procedure-conducting device, the plurality of oral or maxillofacial procedural devices including an ablation probe, a cutting device, a drilling device, and an abrading device, interchangeable in the oral or maxillofacial procedure-conducting device, to register any one of the plurality of interchangeable oral or maxillofacial procedural devices with the fiducial marker upon replacement thereof in the oral or maxillofacial procedure-conducting device;
forming, using a procedure planning device, a virtual procedure plan detailing an oral or maxillofacial procedure at the oral or maxillofacial site, the virtual procedure plan including a procedural route of the oral or maxillofacial procedure-conducting device comprising one of the plurality of oral or maxillofacial procedural devices registered with the fiducial marker, for the one of the plurality of oral or maxillofacial procedural devices to perform the oral or maxillofacial procedure, the virtual procedure plan being in registration with the fiducial marker;
upon the virtual procedure plan detailing the oral or maxillofacial procedure comprising an ablative object removal procedure at the oral or maxillofacial site, placing the ablation probe in the oral or maxillofacial procedure-conducting device;
controlling, using a controller device in communication with the procedure planning device, orientation and distance of the oral or maxillofacial procedure-conducting device along a manipulation path, in correspondence with physical manipulation of the oral or maxillofacial procedure-conducting device along the procedural route by the user for the ablation probe to perform the ablative object removal procedure at the oral or maxillofacial site; and
physically regulating movement of the oral or maxillofacial procedure-conducting device comprising the ablation probe, using a guidance device including a robotic arm engaged with the oral or maxillofacial procedure-conducting device, in response to the controller device in communication therewith and with respect to the fiducial marker, in accordance with the virtual procedure plan, the oral or maxillofacial procedure-conducting device providing tactile feedback to the user, if the physical manipulation of the oral or maxillofacial procedure-conducting device by the user along the manipulation path causes the oral or maxillofacial procedure-conducting device to deviate from the procedural route of the virtual procedure plan.

2. The method of claim 1, wherein the plurality of oral or maxillofacial procedural devices further includes a soft tissue penetrating device or a self-introducing probe having a soft tissue penetrating portion and an object removal portion, interchangeable in the oral or maxillofacial procedure-conducting device.

3. The method of claim 2, wherein:
upon the virtual procedure plan detailing the oral or maxillofacial procedure comprising an object removal procedure or a wisdom tooth removal procedure at the oral or maxillofacial site, replacing the ablation probe with the drilling device, the abrading device, the soft tissue penetrating device, or the self-introducing probe in the oral or maxillofacial procedure-conducting device;
controlling, using a controller device in communication with the procedure planning device, orientation and distance of the oral or maxillofacial procedure-conducting device along a manipulation path, in correspondence with physical manipulation of the oral or maxillofacial procedure-conducting device along the procedural route by the user for the drilling device, the abrading device, the soft tissue penetrating device, or the self-introducing probe in the oral or maxillofacial procedure-conducting device to perform the object removal procedure or the wisdom tooth removal procedure at the oral or maxillofacial site; and
physically regulating movement of the oral or maxillofacial procedure-conducting device comprising the drilling device, the abrading device, the soft tissue penetrating device, or the self-introducing probe, using a guidance device including a robotic arm engaged with the oral or maxillofacial procedure-conducting device, in response to the controller device in communication therewith and with respect to the fiducial marker, in accordance with the virtual procedure plan, the oral or maxillofacial procedure-conducting device providing tactile feedback to the user, if the physical manipulation of the oral or maxillofacial procedure-conducting device by the user along the manipulation path causes the oral or maxillofacial procedure-conducting device to deviate from the procedural route of the virtual procedure plan.

4. The method of claim 1, wherein:
upon the virtual procedure plan detailing the oral or maxillofacial procedure comprising an object-related procedure or a root canal procedure at the oral or maxillofacial site, replacing the ablation probe with the drilling device or the abrading device in the oral or maxillofacial procedure-conducting device;
controlling, using a controller device in communication with the procedure planning device, orientation and distance of the oral or maxillofacial procedure-conducting device along a manipulation path, in correspondence with physical manipulation of the oral or maxillofacial procedure-conducting device along the procedural route by the user for the drilling device or the abrading device in the oral or maxillofacial procedure-conducting device to perform the object-related procedure or the root canal procedure at the oral or maxillofacial site; and
physically regulating movement of the oral or maxillofacial procedure-conducting device comprising the drilling device or the abrading device, using a guidance device including a robotic arm engaged with the oral or maxillofacial procedure-conducting device, in response to the controller device in communication therewith and with respect to the fiducial marker, in accordance with the virtual procedure plan, the oral or maxillofacial procedure-conducting device providing tactile feedback to the user, if the physical manipulation of the oral or maxillofacial procedure-conducting device by the user along the manipulation path causes the oral or maxillofacial procedure-conducting device to deviate from the procedural route of the virtual procedure plan.

5. The method of claim 1, wherein:
upon the virtual procedure plan detailing the oral or maxillofacial procedure comprising an object-related procedure or a procedure for preparing the object for receiving a crown at the oral or maxillofacial site, replacing the ablation probe with the abrading device in the oral or maxillofacial procedure-conducting device;
controlling, using a controller device in communication with the procedure planning device, orientation and distance of the oral or maxillofacial procedure-conducting device along a manipulation path, in correspondence with physical manipulation of the oral or maxillofacial procedure-conducting device along the procedural route by the user for the abrading device in the oral or maxillofacial procedure-conducting device to perform the object-related procedure or the procedure for preparing the object for receiving a crown at the oral or maxillofacial site; and
physically regulating movement of the oral or maxillofacial procedure-conducting device comprising the abrading device, using a guidance device including a robotic arm engaged with the oral or maxillofacial procedure-conducting device, in response to the controller device in communication therewith and with respect to the fiducial marker, in accordance with the virtual procedure plan, the oral or maxillofacial procedure-conducting device providing tactile feedback to the user, if the physical manipulation of the oral or maxillofacial procedure-conducting device by the user along the manipulation path causes the oral or maxillofacial procedure-conducting device to deviate from the procedural route of the virtual procedure plan.

6. The method of claim 1, wherein:
upon the virtual procedure plan detailing the oral or maxillofacial procedure comprising a cutting procedure at the oral or maxillofacial site, replacing the ablation probe with the cutting device in the oral or maxillofacial procedure-conducting device;
controlling, using a controller device in communication with the procedure planning device, orientation and distance of the oral or maxillofacial procedure-conducting device along a manipulation path, in correspondence with physical manipulation of the oral or maxillofacial procedure-conducting device along the procedural route by the user for the cutting device in the oral or maxillofacial procedure-conducting device to perform the cutting procedure at the oral or maxillofacial site; and
physically regulating movement of the oral or maxillofacial procedure-conducting device comprising the cutting device, using a guidance device including a robotic arm engaged with the oral or maxillofacial procedure-conducting device, in response to the controller device in communication therewith and with respect to the fiducial marker, in accordance with the virtual procedure plan, the oral or maxillofacial procedure-conducting device providing tactile feedback to the user, if the physical manipulation of the oral or maxillofacial procedure-conducting device by the user along the manipulation path causes the oral or maxillofacial procedure-conducting device to deviate from the procedural route of the virtual procedure plan.

7. The method of claim 1, comprising imaging the object with respect to the fiducial marker to facilitate registration of the virtual procedure plan with the fiducial marker.

8. The method of claim 1, wherein providing tactile feedback comprises allowing movement of the oral or maxillofacial procedure-conducting device in accordance with the virtual procedure plan and physically preventing movement of the oral or maxillofacial procedure-conducting device deviating from the virtual procedure plan.

9. The method of claim 1, wherein providing tactile feedback comprises vibrating the oral or maxillofacial procedure-conducting device if movement of the oral or maxillofacial procedure-conducting device deviates from the virtual procedure plan.

10. The method of claim 1, comprising engaging the guidance device in communication with the fiducial device such that the guidance device is related with the fiducial marker.

11. The method of claim 10, wherein engaging the guidance device in communication with the fiducial device comprises engaging the guidance device in physical or nonphysical communication with the fiducial device.
